(19) United States
(12) Patent Application Publication
(10) Pub. No.: US 2011/0020299 A1
(43) Pub. Date: Jan. 27, 2011

(54) METHOD AND COMPOSITION FOR THE REGENERATION OF TISSUE WITH THE AID OF STEM CELLS OR BONE MARROW CELLS

(76) Inventor: Augustinus Bader, Klinga (DE)

Correspondence Address: ARENT FOX LLP, 1050 CONNECTICUT AVENUE, N.W., SUITE 400, WASHINGTON, DC, 20036, US

(21) Appl. No.: 12/935,532

(22) PCT Filed: Mar. 24, 2009
(86) PCT No.: PCT/EP09/02135

§ 371 Date: Sep. 29, 2010

(30) Foreign Application Priority Data
Mar. 31, 2008 (EP) 08006238.3

(51) Int. Cl. A61K 35/32; C12N 5/0789; A61P 17/02

(52) U.S. Cl. 424/93.7; 435/325; 435/375

(57) ABSTRACT

The invention relates to a novel polymerisable composition essentially consisting of blood or blood plasma, and also stem cells or bone-marrow cells, and to a method for tissue regeneration with the aid of such compositions. Mixtures of this type are able to polymerise under the influence of endogenous or exogenous polymerisation factors, such as thrombin, calcium ions or cell detritus, to give viscous gels, which are very advantageous for the development and differntiation of the stem cells or bone-marrow cells into tissue-specific cells. Polymers of this type, which, in particular, additionally comprise erythropoietin (EPO), or growth factors with a similar action, exhibit excellent properties on use for tissue regeneration or in the regeneration of bone defects.

The invention relates to a novel polymerisable composition comprising blood, blood platelets or blood plasma, and also stem cells or bone-marrow cells, and optionally further cells from blood, fatty tissue or a tissue which originates from or corresponds to the target tissue to be built up or regenerated. Any desired target tissue can be produced in a targeted manner in a very short time using this blood/stem-cell preparation.

In many injuries and diseases, defects exist in the body, which cannot repair them owing to an evolutionary barrier. The aim of this invention is to allow a “master copy” to become feasible in its production ability for the “engineering” of tissue and also to indicate a method by means of which any desired target tissue can be produced.

Mixtures of this type are able to polymerise under the influence of endogenous or exogenous polymerisation factors, such as thrombin, calcium ions or cell detritus, to give viscous gels, which are very advantageous for the development and differentiation of stem cells or bone-marrow cells into tissue-specific cells.

Polymers of this type, which, in particular, additionally comprise erythropoietin (EPO), analogues or derivatives of EPO (e.g. in carbamylated form) or peptide sequences of EPO and/or thrombopoietin, or thrombin, exhibit excellent properties on use for tissue regeneration or in the regeneration of bone defects. Alternatively, GM-CSF or G-CSF can also be used, alone or in combination with EPO.

The invention furthermore relates to a method for the regeneration and genesis of tissue with the aid of these polymers from blood components, stem cells/bone marrow and preferably factors and substances which promote cell growth and cell differentiation, in particular EPO and biologically active derivatives and/or fragments thereof, and GM-CSF or G-CSF, and optionally vitamins, for example vitamin C, or hormones.

Embryonic, neonatal (from the umbilical cord) and adult stem cells are regarded as carrying the hopes for novel therapy forms with which the regeneration of destroyed tissues and organs is to be made possible. These cells apparently really do have the potential for the repair of destroyed tissue parts, but the underlying mechanisms and the practical applicability with respect to specific tissue are still the subject of controversial debate.

The term “stem cells” encompasses a heterogeneous group of cells which have at least the following two properties in common: stem cells are precursor cells of highly differentiated cells. After division of the stem cells, the daughter cells, according to expert opinion, can either become stem cells again or differentiate in a tissue-specific manner, e.g. into heart, nerve, skin or muscle cells.

Stem cells first occur in early embryonic development. Even the fertilised egg cell (zygote) represents an omnipotent stem cell, which passes through the early embryonic stages and from which all tissue in the human body later forms. Thus, for example, stem cells of the embryonic connective tissue (mesenchymal cells) develop into muscle cells under the influence of certain growth factors during embryogenesis. The further the specialisation of the daughter cells of a stem cell progresses, the greater the range of possible differentiation into different tissue is restricted.

By contrast, other stem cells, the so-called adult stem cells, play an important role throughout life, in particular in tissue regeneration and repair. They maintain the ability of tissues and organs to function by replenishing differentiated cells and replacing damaged or dead cells. For example, stem cells from the bone marrow ensure the replenishment of short-lived blood cells.

Until recently, the prevailing opinion was that adult stem cells are able to produce not only cells of the corresponding organ in which they are found, but also cells of other tissue or organs. For example, the bone marrow has given rise not only to new blood cells, but also cells of various body tissues, such as bones, cartilages, tendons, muscles, liver.

However, the opinion that adult stem cells from the bone marrow can change into any desired differentiated cell is disputed on the basis of recent results, which show that, in mesenchymal stem cells, such a potential for transdifferentiation is only rudimentary or only occurs with certain prerequisites: in many cases in which fully functional specialised tissue cells, e.g. skeletal muscle cells, have actually been produced from mesenchymal stem cells, this has taken place through fusion of the stem cells with cells present which have already fully differentiated. Although experiments show that these cells express, e.g., a number of heart- and skeletal muscle-specific genes if they have been cultivated together with certain growth factor-producing cells, fully functional muscle cells ultimately were not found, although morphological changes were observed in the cells.

Thus, it has hitherto not been possible to carry out stem-cell therapy in humans which results in true regeneration of specific tissue in situ. For example, the transplantation of adult stem cells into the heart results in increased angiogenesis, but apparently not in the development of heart-muscle tissue.

Consequently, starting cells which have already correspondingly specialised or differentiated are very often used in the regeneration of tissue. For example, matrices, such as, e.g., fibrin heterologues, including of autologous origin, but also, e.g., rat-tail collagen, are used for the regeneration of cartilage tissues in accordance with the prior art. Cartilage cells of articular origin are usually introduced into these polymeric matrices. These are obtained after a biopsy from the affected knee joint of a patient and colonised onto these matrices lege artis using ex-vivo expansion methods. Alternatively, the cartilage cells can be cultivated directly in the alginate, fibrin or collagen matrices of animal origin in a known manner and transplanted after a cultivation time of a few weeks. Similar methods are also known for the production of other tissue-specific cells.

A disadvantage of these methods is that the extended precultivation times that are necessary result in de-differentiation processes of the selected specific cells, which then take on undesired properties. For example, misdirected cartilage-cell differentiation processes of this type favour the formation of the undesired fibrotic cartilage instead of hyaline cartilage.

A further essential disadvantage of the usual methods to date consists in that the polymeric matrices used today do not favour the re-formation and transformation of the tissue and trigger undesired cell activation. In addition, immunological incompatibilities not infrequently also arise for the recipient.

The said disadvantages can now be avoided in accordance with the invention through the use of stem cells instead of differentiated cells, where stem cells are not incorporated, as usual to date, directly into the target tissue to be regenerated, but instead by means of a matrix which essentially consists of polymeric blood or blood plasma or polymerisable blood platelet concentrates or preparations in which the stem cells or precursor cells thereof have been embedded before the polymerisation.

Thus, it can be shown, surprisingly, that the use of polymers of this type, in particular containing adult stem cells, based on blood, or cellular constituents thereof (red and white blood corpuscles, blood platelets) or non-cellular constituents (proteins, lipids, sugars, etc.) produces significantly higher yield and quality of the differentiated special cells obtained than the use of the known methods as described above, including the methods which use tissue cells as starting cells.

Surprisingly, this effect can also be observed compared with corresponding blood preparations which have not been polymerised in advance. Polymerised blood or blood plasma thus exerts a very positive effect, in contrast to liquid blood samples, on the stem cells with respect to their ability to differentiate into tissue-specific cells and to multiply in differentiated form.

In particular, the polymerisation promoters, such as thrombin, Ca++ or synthetic or biological matrix fragments, such as, e.g., peptides which contain collagen sequences or also RDG sequences, are responsible not only for the polymerisation, but also for the biological effect in the 3D structure of the blood, the plasma structure, or the structure of the blood-platelet preparation after polymerisation.

The reason for this is that a series of growth factors which have a direct stimulating action for the stem cells are thereby released. These include EGF, but also TGF beta. Besides these factors, however, cell-membrane constituents, matrix constituents, cell-contents constituents and complex mineral elements (Na, K, Cl, Mg, zinc) represent substance contamination which does not usually arise in normal synthetic materials. In addition, fibrin polymers are also unable to do these jobs in view of the complexity of the micromedium.

It has furthermore been found in accordance with the invention that this effect of polymerised blood arises, in particular, in the presence of factors which promote the release, multiplication or differentiation of stem cells. These are not only the usual hormones, vitamins or growth factors employed in stem-cell production methods, such as, for example, GM-CSF, G-CSF, but also, surprisingly and particularly, also erythropoietin (EPO), analogues, carbamylated forms or also peptide fragments thereof which contain sequences of the natural substances.

This is because it has been found in accordance with the invention that an artificial and strengthened wound medium is created in the 3D structure of the blood gel according to the invention in combination with the stem cells or progenitors, which triggers a cascade reaction in relation to the stem cells in combination with the oxygen deficiency which occurs under these conditions and the local occurrence of released acute-phase cytokines, such as, e.g., interleukin-6 (IL-6), interleukin.1 (IL-1) and tumour necrosis factor (TNF). The co-stimulation with EPO/derivatives or analogues gives rise here to a significant permissive effect, which allows the actual effector cells (stem cells and progenitors) to translate into tissue differentiation.

A further advantage of the gel structure and the conversion, inducible thereby, from a liquid (sol) to a solidified (polymerised) gel structure lies in the modelling ability in a wound cavity or wound defect and in the general suitability for topical application through the adhesive action of the compounds. Stem cells can thus be introduced highly effectively into the vicinity of a wound base, and defects can thus be filled in a simple manner.

The trauma situation can also be locally limited by this application cocktail according to the invention in the case of chronic disease forms, such as, e.g., a transverse injury in the chronic state, in which the local trauma situation has already subsided, or also in the case of cosmetic interventions in the area of the skin for scar correction or for the construction of filling defects, in breast reconstruction with repeated subcutaneous injections or also topically at intervals of 3-4 days. This creates an ideal environment, in a microarea, for stem-cell stimulation for initiation of location-specific tissue development. Due to the permeability of the 3D construct, hormonal and paracrine signals from the surrounding area may additionally act on the stem cells and stimulate the latter.

In contrast to the natural situation in a wound, the combination of these factors triggers an augmentation of the effects, which can cause accelerated wound healing of up to about 50%. A further advantage of the gel structure, besides the autologous basis, is the full complementability with synthetic components (BMPs, PDGF, EPO, GCSF, GM-CSF or synthetic matrix components.

Blood polymers, bone-marrow polymers or blood-platelet concentrates which comprise stem cells and preferably additionally EPO and/or GM-CSF or G-CSF, and which are introduced into corresponding tissue or into corresponding (defective) bone structures, exhibit to a high degree an increased ability to effect the generation of functional differentiated cells of the target tissue.

A further advantage becomes evident here, namely that, in the case of defect sizes or also defect types which no longer alone enable restitutio ad integrum or restoration of the original tissue, filling materials which are similar to the target tissue and thus represent a quasi “master copy” for the remodelling process of the stem cells activated in the application cocktail, can be incorporated here. The materials of the master copy can be, e.g., of a mineral nature for the bones or dentin substitute in the case of teeth. Synthetic RGD analogues, collagen peptides (preferably from animal collagens) can be admixed with the natural composition of the bone and brought to gel polymerisation on site in the wound or defect.

The invention thus relates to a polymer comprising preferably autologous and essentially human or animal blood or blood constituents with all its degeneration constituents, such as cellular detritus, which is distinguished by the fact that it comprises stem cells or bone-marrow cells and at least one substance which effects or promotes or increases the release, multiplication or differentiation of the stem cells or bone-marrow cells.

Corresponding substances which promote the growth and differentiation of stem cells are growth hormones, such as HGH or cytokines, such as, e.g., interleukins, interferons, TNFa or G-CSF or GM-CSF.

In particular, erythropoietin (EPO) or one of its biologically active analogues, derivatives or fragments is suitable. Erythropoietin (EPO) is a glycoprotein hormone which controls the formation of the erythrocytes from precursor cells in the bone marrow (erythropoiesis). In the process, EPO binds to its receptor (EPO-R), which is expressed in all haematopoietic cells.

In recent years, diverse authors have reported that EPO also exerts a non-haematopoietic action, and the EPO-R is correspondingly also expressed by certain non-haematopoietic cells. Thus, stimulation of nerve cells, neuronal cells of the brain and endothelial cells by EPO is reported, in some cases associated with direct expression of the haematopoietic EPO receptor.

In other cases, the presence of a further, non-haematopoietic receptor is suggested. In particular, the non-haematopoietic action of erythropoietin (EPO), which has not yet been known for very long, in connection, for example, with the stimulated formation and regeneration of endothelial and tissue cells is increasingly being regarded as important.

Thus, WO 2004/001023 describes, inter alia, the use of EPO and TPO for the stimulation of neovascularisation and tissue regeneration and improvement in wound healing, e.g. after operations or injuries.

WO 2005/063965 teaches the use of EPO for the targeted, structurally controlled regeneration of traumatised tissue, in which not only is endothelial cell growth stimulated, but also parenchymal regeneration and the formation of wall structures is promoted, meaning that coordinated three-dimensional growth for the development of a functional tissue, organ or parts thereof takes place. Erythropoietin, and EPO derivatives or also EPO mimetics thus appear to be suitable, on systemic administration, for initiating and controlling the re-formation and regeneration of the affected tissue in a targeted manner in the case of injuries to the skin, the mucous membrane, in the case of open skin and flesh wounds or also in the case of skin irritation due to burns or scalds, and ultimately for being able to promote and accelerate healing.

WO 2005/070450 and further papers by the inventors in question describes the use of EPO in the regeneration of vessels and tissue with a weekly dose of less than 90 IU/kg of body weight, inter alia also for the area of wound care. The aim of this is to achieve a situation in which blood formation in the bone-marrow area is stimulated less, but, according to more recent teaching, as outlined, activation of endothelial cell progenitors in the blood area is possible. Activation of endothelial cell precursor cells in the blood, but also in the tissue, and the development of endothelial cells, which form the innermost cell layer of blood vessels, has been associated with an improvement in vascularisation, and it is thought that tissue regeneration is thereby also facilitated. In the meantime, this has been confirmed in clinical trials for burn wounds.

It has now been found in accordance with the invention that the effect already promoted by blood polymers on stem-cell development and differentiation can be increased further by about 10-50% if the corresponding polymers additionally comprise EPO, where the EPO dose is between 50 and 500 IU/kg of body weight, preferably between 100 and 300 IU/kg of body weight. The reason lies not only in the 3D structure made available, but also in the surprising synergistic effects, which have to date been interpreted conversely in accordance with the prior art. Thus, it is known that greater layer thicknesses cannot be achieved in tissue engineering since the oxygen deficiency which arises limits tissue formation to a few microns of layer thicknesses (usually 100-500 μm). In addition, high-purity cell populations are produced, which should not contain any dead cells.

However, the totality of the combination represents a surprisingly positive effect for the stem cells introduced into the blood polymer, where the polymerisation, the increased layer thicknesses, the cellular degradation of, e.g., the mononuclear cells and activation thereof under ischaemic conditions promotes acute-phase reactions on the heterogeneous stem cells, which initiates an explosive trigger effect in combination, in particular, with EPO. If selective “copyable” components of the extracellular medium are then also added, a true “remodelling” effect, which results in de novo tissue formation, arises in accordance with the invention. This tissue formation may also arise if the type and size of the defect would not facilitate healing alone.

The invention thus relates, in particular, to a blood polymer comprising stem cells or bone-marrow cells, which additionally comprises erythropoietin (EPO) in a suitable dose. The use of polymers of this type has the effect, depending on the tissue-specific application, that tissue regeneration takes place 10-50% more quickly and in a qualitatively/functionally improved manner compared with the conventional methods, which are based on the use of tissue cells which have already specialised.

The EPO-containing stem-cell polymers according to the invention based on preferably autologous blood or blood plasma or blood platelets may additionally comprise substances which promote growth and differentiation, in particular GM-CSF, G-CSF or TNFalpha.

In accordance with the invention, EPO and biologically active derivatives thereof in combination with a polymerisation process of blood/bone marrow/blood-platelet concentrate, plasma, red and/or white blood corpuscle fraction enables the survival and further development of stem cells or precursor cells thereof as well as tissue-specific activation and, in the case of trauma, specific regeneration of tissue.

The invention is based on the fundamental discovery that blood or blood plasma in combination with stem cells adopts a viscous consistency, and the 3D structures apparently formed thereby favour the development of the stem cells. The introduction of exogenous gel formers into the liquid mixture of blood/blood plasma, stem cells, EPO, etc., may augment this effect further, enabling the formation of gels or a polymer of different strength and having good processing properties. Simple gel formers which are also very successful in accordance with the invention have proven to be, for example, thrombin with and without calcium ions, or protamine, which can be added to the still-liquid mixture.

Specific application methods can be used therefrom by, e.g., transferring the mixture comprising stem cells in a syringe while still liquid into a single application cannula via forced mixing with a 2nd syringe containing the “master copys” and only polymerising the mixture at the site of application.

An adequate polymerisation effect in the sense discussed above, which has a favourable effect on the development of stem cells or precursor cells thereof, is also obtained by addition of cell detritus to the mixture to be polymerised. Cell detritus is formed by from cell-containing samples by the death of cells, such as mononuclear cells, red and white blood corpuscles (leucocytes), blood leaflets or stem cells, precursor cells, fibroblasts, endothelial cells, connective-tissue cells, cartilage cells and macrophages, owing to deficient supply, with substances which have advantageous properties for the polymerisation or the quality of the polymer advantageously being released. Thus, calcium ions are also released endogenously, making addition of exogenous calcium entirely or partly superfluous. Cell detritus of autologous origin is advantageously added to the mixtures to be polymerised.

The invention thus also relates to a corresponding blood-containing polymer which comprises endogenous or exogenous substances which promote fixable and conformative structures of the stem cells and thus development thereof. Substances of this type can be thrombin, calcium ions, cell detritus of various types of cell, preferably of autologous cells, biological collagens or fragments thereof, constituents of the extracellular matrix, fibrin, fibrin adhesives or other gel-forming substances.

The polymerisation may alternatively or additionally be effected by other natural or synthetic polymer formers, such as, for example, gel-forming swellable polysaccharides, such as hydroxyalkycelluloses and/or caboxyalkylcelluloses, or synthetic polymer formers based on acrylates, such as, e.g., (poly)methacrylate, (poly)methyl methacrylate, polyacrylamide, (poly)ethoxyethyl methacrylate.

It has furthermore been found that the addition of lyophilised blood to the mixture to be polymerised improves or augments the polymerisation stability of the polymer and the property thereof in relation to its tissue-regenerating effect. In addition, tacky proteins based on the scallop in native or synthetic form can be admixed. It is likewise possible to add serotonins, which not only stimulate neuronal progenitors, but also support an adhesive action.

With the aid of thrombin, calcium ions and/or cell detritus and/or other suitable gel formers and/or lyophilised blood, a number of possibilities are available for varying and modifying the polymerisation properties of the mixture comprising blood/blood plasma and stem cells and thereby surprisingly influencing stem-cell development and differentiation.

In accordance with the invention, the stem cells need not be isolated separately. It is also sufficient to use bone marrow from an individual directly and to mix this with preferably autologous blood or blood plasma, to concentrate this, if necessary, with respect to the cellular components by centrifugation, filtration or sedimentation and to add thrombin, calcium ions and/or cell detritus, matrices (minerals, peptides, sugars, lipids and combinations thereof) and preferably EPO, and to bring the mixture to gel formation. A gel of this type can be employed directly for a very wide variety of applications.

Thus, the invention relates to a corresponding polymer which can be employed in vivo for the regeneration of tissue or bone, in particular of traumatised tissue and/or inter alia bone.

In particular, the invention relates to the use of a corresponding polymer for the regeneration of traumatised tissues or bone structures, where the polymer is intended to be, introduced into the affected traumatised area or into its immediate vicinity, or used for filling defects.

It has been found that such a polymer according to the invention can particularly advantageously be employed in combination with bone substitute materials in the case of bone defects. In this case, the mixture just before onset of polymerisation and the still not fully polymerised bone substitute material can be introduced simultaneously into the bone defect and mixed there in order to polymerise thereafter. This is particularly advantageous for, e.g., nanocrystalline hydroxylapatite or other nanocrystalline mineral or biological material (proteins, peptides, lipids, sugars, plastics), which is either in dry or hydrogenated form. Due to this pulverulent or pasty material property, the ability to be colonised by stem cells is restricted or even to the disadvantage of the biological materials. Thus, it has been found that material of this type (e.g. “Ostim”) cannot be colonised.

It is nevertheless and surprisingly possible in accordance with the invention to achieve a high stem-cell density in the nanocrystalline material regarded as uncolonisable.

After an in-vivo implantation, this results in an acceleration of bone development by about 50% even without the addition of EPO. On additional introduction of EPO in topical form, the development of bone can be additionally accelerated by 10-20%. Alternatively, EPO, derivatives thereof, or peptide fragments can also be added to the scaffold material as a powder. This has the advantage of better storage ability.

Alternatively, e.g. on use of two application containers in the form of syringes, the EPO can be prepared in lyophilised form in the syringe, which accommodates the blood, bone-marrow stem-cell mixture. Furthermore, matrix (scaffold) components of autologous origin (lyophilised blood or plasma, fibrin, thrombin) and of synthetic origin (peptide fragments of matrix proteins, self-aggregating peptide structures (RADA), phosphatidylcholine, sphingolipids, lecithin, HDL (high density lipoproteins), and glucose, glucosamines, glucosamine sulphates, hyaluronic acid, chitosan, silk proteins, adhesive proteins from the scallop, collagens and fragments thereof, as well as peptides may likewise be present on this side (syringe 2).

This application method enables the stem cells to be distributed optimally and a heterogeneous “scaffold” structure, into which bone tissue is able to grow directly from the outside via entry pathways and can replace the latter successively, to be obtained within the, e.g., bone substitute matrix. The interconnecting porosities thereby formed are thus permeated ab initio with stem cells in gelatinous structures. This creates optimum growth conditions in spite of very large layer thicknesses.

Contrary to the conventional teaching, the addition of EPO, GM-CSF or G-CSF, but in particular EPO, in this situation results in particularly efficient stimulation of stem cells or progenitors in optimised media. This makes it possible to omit corresponding expansion methods of progenitors outside the application site (practice, operating theatre) and to carry out stem-cell therapy at the same time as the actual surgical intervention (intra-operatively). This has significant regulatory and economic advantages (cost reduction) and means a minimisation of risk and a significant improvement in quality.

In the case of particularly large defects, however, pre-expansion in the stem-cell gel may be advantageous. In accordance with the invention, however, this cultivation can then take place immediately in the 3D blood gel or plasma gel. For this purpose, the polymerisation can also be initiated in vitro in order to create the wound medium in vitro which to initiate the ideal growth conditions for the stem cells/progenitors in a paracrine stimulation medium with oxygen deficiency owing to the increased layer thicknesses from a thickness of several millimetres to a thickness of several centimetres in diameter. In the case of the intra-operative variant, even a few seconds to a few minutes are sufficient to initiate the requisite stimulation processes of the stem cells simultaneously (IL-6, EPO, GM-CSF, G-CSF, matrix).

The invention thus relates to the use of a polymer in this respect in combination with a bone substitute material, in particular for the regeneration and reconstruction of defective, traumatised or diseased bone structures or bone tissues in vitro, in vivo or ex vivo.

The mineral components of the bone substitute material should be suitable here for supporting the filling of the bone defect. Suitable bone substitute materials which may be mentioned are, for example, hydroxylapatite, tricalcium phosphate and the like.

The polymers according to the invention are suitable for an extremely wide variety of medical uses in which tissue or bone defects have arisen due to traumatisation or disease. In particular, they can also be employed in the dental area in the case of tooth and jaw defects, but also in the topical area in the case of injuries to the skin or mucous membrane.

The invention furthermore relates to a method for the preparation of a corresponding polymer comprising human or animal blood as well as human or animal stem cells or bone-marrow cells, in which said components are mixed with one another in the liquid state, and this mixture is polymerised or solidified by means of a gel former, in particular by addition of calcium ions, thrombin, fibrin or constituents of the extracellular matrix of biological origin or cell detritus and optionally additionally lyophilised blood, where, in a particular embodiment, one or more substances which effects or promotes the release, multiplication or differentiation of the stem cells or bone-marrow cells are added to the liquid mixture before polymerisation.

The invention furthermore relates to a method for the regeneration of tissue or bone structures in vitro or ex vivo, comprising the following steps:

(i) provision of isolated stem cells or bone marrow

(ii) introduction of the said cells into a blood, blood-platelet or blood-plasma sample

(iii) polymerisation of the sample from (ii) and

(iv) cultivation of the cells in a suitable medium which initiates and/or promotes the growth and differentiation of the cells into the desired tissue.

The invention additionally relates to a method for the regeneration of tissue or bone structures, comprising the following steps:

(i) provision of isolated stem cells or bone marrow

(ii) introduction of the said cells into a blood or blood-plasma sample or direct use of bone marrow and mixing with EPO, GCSF, GM-CSF

(iii) polymerisation of the sample from (ii) with, e.g., Ca++ or prothrombin, protamine and optionally with further factors, such as “copying materials” and differentiation factors of synthetic or natural origin.

(iv) introduction of the sample directly into the injured, diseased, defective of degenerated region of the body.

The above-mentioned differentiation factors can be TGFbeta or paratho one (cartilage formation), or vitamin C (for induction of neuronal sprouting).

As an important addition for improving “tissue engineering”, fragments or particles of the target tissue are also integrated. These ideally have a thickness of about 100-200 μm and a diameter of about 200-300 μm. Larger or smaller fragments are possible. The comminution is ideally carried out using a scalpel or sharp knife. The advantage is that this preparation process only takes very little time. This entire process can therefore be carried out in a preferred form at the same time as the actual operation.

As an alternative to the complete mixture of the cells present in bone marrow, it would also be possible to employ isolated stem cells or progenitor populations, such as CD31, CD71 and CD134, and CD90-positive cells.

In particular, the subject-matter is methods in vivo, in vitro and ex vivo in which the polymerisation of the blood or blood-plasma sample is carried out by addition of calcium ions and/or a natural or synthetic gel-forming substance, and/or cell detritus.

The subject-matter is furthermore methods in vivo, in vitro and ex vivo in which growth factors and/or hormones and/or substances which promote growth or differentiation and/or cells are added, in particular selected from the group consisting of EPO, GM-CSF, G-CSF, GH, connective-tissue cells, fibroblasts, macrophages, cell detritus.

The following examples are intended to explain the invention further without restricting it. In particular, the specific method steps, method parameters, substances, tissue samples and applications mentioned are non-limiting and may be replaced by other method steps, method parameters, substances, tissue samples and applications with the same action if the person skilled in the art readily sees a reason for this.

### EXAMPLE 1

### Therapy of Nerve Injury, In Particular Transverse Injuries

For acute transverse injuries, decompression of the affected nerve area must be carried out.

In accordance with the invention, erythropoietin is added here in combination with bone marrow and blood and applied to the wound area like an ointment or gel.

Ideally, the gel has a layer thickness of about 1-2 mm and contains about 100,000 cells.

In the case of relatively large defects in the spinal canal, the bone marrow can be mixed with lyophilised blood or alternatively with a comminuted collagen sponge. This promotes the formation of a relatively large mass and a guide structure. A blood-platelet concentrate in native or freeze-dried form can likewise be added to this mass. At the same time, vitamin C (10-20 mg) is added to the whole.

### EXAMPLE 2

### Bone Development In Combination With Minerals

One of the essential advantages of the invention is that finished products, such as, e.g., bone substitute materials in aqueous solution, which are essentially uncolonisable, can nevertheless be provided with stem cells in a very effective manner.

To this end, the conventional bone substitute material is used in a first syringe and the blood/stem-cell mixture, with and without EPO, with or without calcium ions, cell detritus, etc., is used in a second syringe in a tandem syringe system. In detail, a lyophilisate of EPO (e.g. 10,000 IU for a person weighing 70 kg), additionally Ca++ (1 mg in crystalline form or 1 mol/l) is placed in an empty syringe. 1-2 ml of bone marrow and in addition 1 ml of blood-platelet concentrate are sucked into this syringe. The mixture in this second syringe is then applied to a bone defect in parallel at the same time as the still-deformable bone substitute material in the first syringe via a forced mixing screw from the tandem syringe system and mixed and brought to polymerisation on site.

The second syringe may additionally contain synthetic or biological collagens or fragments thereof, constituents of extracellular matrix, or other substances, such as, e.g., RGD peptides. In addition, BMP or blood platelets or blood concentrates in lyophilised or native form may be admixed as concentrate.

### EXAMPLE 3

### Filling of A Bone Cyst

For the preparation of a pasty and flexible mass for filling cavities, the following procedure is followed: about 10 ml of bone marrow are introduced into a tube/syringe containing lyophilised erythropoietin, lyophilised blood plasma and nanocrystalline hydroxylapatite or tricalcium phosphate.

Within a short time, a highly pasty, tacky mass forms, which is introduced into the defect area of the bone using a spatula or via a cannula.

Furthermore, synthetic components of the extracellular matrix, such as fibronectin or collagens, are added, causing growth zones for the integration of the cellular systems to form or improve more quickly.

### EXAMPLE 4

### Regeneration of Heart Muscle Tissue

About 500 ml of peripheral blood is taken and centrifuged at 50 g for 5 min. The supernatant is then centrifuged again at 800 g for 5 min, and the pellet is combined with the previous pellet. All cells obtained in this way from the peripheral blood are taken up in a syringe which [lacuna] 250 units/kg of body weight of EPO, and the stem cells are ready for implantation after an incubation time of 5 min and activation of the EPO receptors. Alternatively or in combination, 5-10 ml of bone marrow can be introduced into the syringe with the lyophilised EPO.

An analogous procedure is followed in order to apply stem cells to the injured area after muscle fibre tears. About 1-2 ml per injection site are used for this purpose.

### EXAMPLE 5

### Regeneration of Mammary Gland Tissue

Centrifuged-off bone marrow is added to 2-3 ml of fatty tissue from the knee-joint area and applied topically with 250 units/kg/body weight. After an incubation time of about 10 sec to 5 min, the stem cells are ready for injection. The injection solution may comprise lyophilised thrombin and Ca++. The stem-cell mixture is employed analogously for use for the development of subcutaneous fatty tissue and for wrinkle reduction and rejuvenation of the skin. After centrifugation, thrombin and/or Ca++ are added to the stem cells from the bone marrow (from 10 ml) or peripheral blood (from 100 ml), which are administered subcutaneously in a volume of about 500 μl per injection site. This procedure can be repeated every 3-4 days or weekly.

### EXAMPLE 6

### Regeneration of Intervertebral Discs

In the case of acute or chronic intervertebral disc injuries, decompression of the affected nerve area must be carried out. In the case of resection of a sequester, this nerve area is decompressed. Extremely small tissue fragments are produced from this sequester material by mechanical comminution. The bone marrow (2 ml), either in native or concentrated form, is added thereto (±0.5 ml), and at the same time lyophilised EPO, GM-CSF or G-CSF in lyophilised form is added in accordance with the usual dosage recommendations in relation to the body weight. However, the administration here is not systemic, but instead preferably topical. 1-2 ml of blood-platelet concentrate, preferably in lyophilised form, can be added to the entire mixture.

In accordance with the invention, erythropoietin is added here in combination with bone marrow and/or blood and injected onto the intervertebral disc area like a gel by means of a sterile syringe.

The gel ideally contains about 100,000-1,000,000 cells, but more or fewer is possible.

In the case of relatively large defects in the intervertebral disc area, the bone marrow can be mixed with lyophilised blood or alternatively with a comminuted collagen sponge. This promotes the formation of a relatively large mass and a guide structure. A blood-platelet concentrate in native or freeze-dried form can likewise be added to this mass. At the same time, vitamin C (10-20 mg) is added to the whole.

### EXAMPLE 7

### Regeneration of Cartilage Tissue

In the case of acute or chronic cartilage injuries and arthroses, the polymer is introduced into a slightly opened joint region by means of a soft, e.g. silicone, tube just before solidification, so that the polymerisation can take place on site. In the case of resection of sequesters, they can be converted into extremely small tissue fragments by mechanical comminution. The bone marrow (2 ml), either in native or concentrated form, is added thereto (±0.5 ml), and at the same time lyophilised EPO, GM-CSF or G-CSF in lyophilised form is added in accordance with the usual dosage recommendations in relation to the body weight. However, the administration here is not systemic, but instead preferably topical. 1-2 ml of blood-platelet concentrate, preferably in lyophilised form, can be added to the entire mixture.

In accordance with the invention, erythropoietin is added here in combination with bone marrow and/or blood and injected onto the intervertebral disc area like a gel by means of a sterile syringe.

The gel ideally contains about 100,000-1,000,000 cells, but more or fewer is possible.

In the case of relatively large defects in the cartilage area, the bone marrow can be mixed with lyophilised blood or alternatively with a comminuted collagen sponge. This promotes the formation of a relatively large mass and a guide structure. A blood-platelet concentrate in native or freeze-dried form can likewise be added to this mass. At the same time, vitamin C (10-20 mg) is added to the whole. This also enables very large joint areas to be supplied. In the upper joint areas towards the joint gap, the blood-platelet concentrate is increasingly employed in a layered manner. In certain cases, TGFbeta and/or parathormone can be employed in order to promote cartilage formation.

### EXAMPLE 8

### Regeneration of Tendon Tissue And Meniscus

In the case of injured tendon tissue (e.g. Achilles tendon in humans and horses) or meniscus, concentrated bone marrow to which EPO and GCSF or GM-CSF have been added is injected into the injured area. Dehiscences are ideally approximated in the usual manner by means of a seam. Tissue fragments are produced from torn-off regions, introduced into the polymerisation gel and re-injected therewith into and around the dehiscence area.

The regeneration of inner ear ossicles or of the retina is carried out analogously.

### EXAMPLE 9

### Improvement of Implant Surfaces In Order To Prevent Capsular Fibroses In Breast Prostheses

One of the major problems in the implantation of breast prostheses is that capsular fibrosis develops around the implant. In accordance with the invention, a microstructure is applied to the implant surface, which enables an ingrowth behaviour of stem cells on these surfaces. This microstructure has cavities having a lower diameter of ideally 4-5 μm and a diameter for larger cavities of 25-250 μm. This cavity structure can ideally be connected to connecting channel structures, which enable the self-organisation of a vascular bed. Direct colonisation by bone marrow is carried out in the cavities, ideally after fresh removal from the bone marrow. In order to concentrate the cells, centrifugation at about 30-50 g can facilitate gentle enrichment. In order to initiate polymerisation and re-formation of tissue, thrombin is added.

The cells from the bone marrow mixed with blood are brought to gel formation and polymerisation in the microstructures by addition of thrombin. The thrombin mixture/stem-cell mixture in combination with blood has a particularly regeneration-friendly action. In combination with the regional wound area at the implantation site, this triggers growth processes which result in approximately 50% reduced fibrosis. In combination with the advantageously topical application of erythropoietin, a regional stimulation effect arises, which results in direct activation of introduced and also local progenitors. These include adipogenic and gland progenitors. The surface markers are CD 90, SCA1, CD 71 are found on these progenitors. Capsular fibrosis also plays an important role, complicating the course of healing, in the implantation of meshes after hernias or in the closure of stomach-wall defects.

Further implants microstructured surfaces are provided analogously with the stem-cell blood gel, with or without EPO/derivatives/analogues, which polymerises on site.

The treatment of hip prostheses or other joint prostheses is carried out analogously.

### EXAMPLE 10

### Dental Implants

The regeneration of dentin after root treatment can be achieved in accordance with the invention by the introduction of a nanostructured stem-cell blood gel comprising hydroxylapatite or tri-calcium phosphate, with or without EPO and also with or without stem cells. The granule size or mineral size here is ideally 5-100 μm, where upward enlargements mean impairments in the flow properties in the drill channel.

### EXAMPLE 11

Other implants can be treated analogously as described in the examples mentioned above, such as, e.g.: cardiac pacemakers, stomach-wall meshes, tracheal replacement, vascular replacement, or heart-valve replacement.

### EXAMPLE 12

### Regeneration of Burn Wounds, Decubitus Or Diabetic Ulcer, Infected Wounds

In the case of acute or chronic skin injuries, a stem-cell gel is prepared as follows and positioned topically after cleaning of the wound base.

Extremely small skin fragments are produced as described in principle above by mechanical comminution from areas of uninjured skin. The bone marrow (2 ml), either in native or concentrated form, is added thereto (±0.5 ml), and at the same time lyophilised EPO, GM-CSF or G-CSF is added in accordance with the usual dosage recommendations in relation to the body weight. However, the administration here is not systemic, but instead preferably topical. 1-2 ml of blood-platelet concentrate, preferably in lyophilised form, can be added to the entire mixture.

In accordance with the invention, erythropoietin is added here in combination with bone marrow and/or blood and applied to the wound area like a gel by means of a sterile syringe or a spatula. The gel ideally contains about 100,000-1,000,000 cells/10 cm², but more or fewer is possible.

In the case of relatively large defects, the bone marrow can be mixed with lyophilised blood or alternatively with a comminuted collagen sponge. This promotes the formation of a relatively large mass and a guide structure. A blood-platelet concentrate in native or freeze-dried form can likewise be added to this mass. At the same time, vitamin C (10-20 mg) is added to the whole

1. A polymer comprising human or animal blood, blood plasma or blood-platelet concentrate, wherein the polymer further comprises stem cells or bone-marrow cells and at least one substance which effects or promotes the release, multiplication or differentiation of the stem cells or bone-marrow cells.

2. The polymer according to claim 1, wherein the promoting substance is a growth factor, a hormone or a cytokine.

3. The polymer according to claim 2, wherein the promoting substance is erythropoietin (EPO), GM-CSF, G-CSF or a biologically active derivative or fragment thereof.

4. The polymer according to claim 3, wherein the promoting substance is EPO or a correspondingly biologically active derivative or fragment thereof.

5. The polymer according to, claim 1, wherein the polymer additionally comprises lyophilised blood or blood plasma.

6. The polymer according to claim 1, which additionally comprises a natural or synthetic polymer and/or calcium ions and/or thrombin and/or protamine.

7. The polymer according to claim 6, which is fibrin or an adhesive based on fibrin.

8. The polymer according to claim 1, wherein autologous stem cells or bone-marrow cells are employed.

9. The polymer according to claim 1, wherein the polymer comprises cell detritus and/or further autologous or heterologous cells from a target tissue, selected from the group consisting of fibroblasts, endothelial cells, connective-tissue cells, cartilage cells and macrophages.

10. A pharmaceutical composition comprising the polymer according to claim 1 in an amount effective for the regeneration of tissue or bone.

11. A method for the preparation of a medicament for the regeneration of tissue, the method comprising combining the polymer of claim 1 with a pharmaceutically acceptable excipient.

12. The composition of claim 10, in a form for the topical treatment of injured skin.

13. The composition of claim 10 wherein the tissues or bone structures are traumatized and wherein the composition is in a form that can be introduced into the affected traumatised area or into its immediate vicinity.

14. The composition of claim 13, further comprising a bone substitute material.

15. (canceled)

16. The composition of claim 10 further comprising mineral components.

17. A method for the regeneration of tissue or bone structures in vitro or ex vivo, comprising the following steps:
(i)provision of isolated stem cells or bone-marrow cells
(ii) introduction of the said cells into a blood or blood-plasma sample or of a blood-platelet concentrate
(iii) polymerisation of the sample from (ii)
(iv) cultivation of the cells in a suitable medium which initiates and/or promotes the growth and differentiation of the cells into the desired tissue.

18. The method according to claim 17, wherein the polymerisation of the blood or blood-plasma sample or of the blood-platelet concentrate is carried out by addition of calcium ions and/or a natural or synthetic gel-forming substance and/or cell detritus and/or thrombin.

19. The method according to claim 17, wherein lyophilised blood or blood plasma is added.

20. The method according to claim 17, wherein growth factors and/or hormones and/or substances which promote growth or differentiation and/or cells from the target tissue are added.

21. The method according to claim 20, wherein one or more of the substances, factors or cells are selected from the group consisting of EPO, GM-CSF, G-CSF, GH, connective-tissue cells, fibroblasts, macrophages and cell detritus.

22. A method for the preparation of a polymer comprising human or animal blood, blood plasma or blood leaflets human or animal stem cells or bone-marrow cells, wherein in said method, said components are mixed with one another in the liquid state, and this mixture is polymerised by means of a gel former.

23. The method according to claim 22, wherein lyophilised blood is additionally added.

24. The method according to claim 22, wherein one or more substances which effects or promotes the release, multiplication or differentiation of the stem cells or bone-marrow cells are added to the liquid mixture before polymerisation.

25. The method of claim 22, wherein said mixture is polymerized by the addition of calcium ions, thrombin, fibrin or constituents of the extracellular matrix of biological origin.
